(19) United States
(12) Patent Application Publication
(10) Pub. No.: US 2015/0279066 A1
(43) Pub. Date: Oct. 1, 2015

(54) Method for Processing Images to Remove Bright-Burn Artifacts and X-Ray Device

(71) Applicant: Michael Stark, Forchheim (DE)

(72) Inventor: Michael Stark, Forchheim (DE)

(21) Appl. No.: 14/665,358

(22) Filed: Mar. 23, 2015

(30) Foreign Application Priority Data
Mar. 28, 2014 (DE) 102014205841.4

(51) Int. Cl. G06T 11/00; A61B 6/00

(52) U.S. Cl. CPC G06T 11/008; A61B 6/5258; A61B 6/4233

(57) ABSTRACT

A method for processing an X-ray image of an object under examination taken with an X-ray detector with a plurality of pixel sensors is provided to remove bright-burn artifacts. At least one substantially artifact-free offset image of the X-ray detector without the application of X-rays is provided. At least one dark image with the X-ray detector without the application of X-rays is recorded. The at least one dark image is corrected by the at least one artifact-free offset image for determining at least one offset-corrected first correction image with afterglow artifacts. At least one second correction image for correcting bright-burn artifacts from the first correction image with afterglow artifacts is calculated using a, in particular predetermined, correlation between afterglow artifacts and bright-burn artifacts. At least one X-ray image of an object under examination is recorded with the X-ray detector, and the at least one X-ray image is corrected with the at least one second correction image to remove bright-burn artifacts.

### RELATED CASE

This application claims the benefit of DE 102014205841.4, filed on Mar. 28, 2014, which is hereby incorporated by reference in its entirety.

### BACKGROUND

The present embodiments relate to a method and x-ray device for processing an X-ray image taken with an X-ray detector with a plurality of pixel elements to remove bright-burn artifacts.

Known in digital X-ray imaging are, for example, X-ray detectors, in particular flat-image detectors, with active readout matrices with direct or indirect conversion of the X-rays. The active matrix is divided into a plurality of pixel sensors. In the case of a so-called directly converting X-ray detector, the incident X-rays are converted directly into electrical charge in a converter layer. In the case of an indirectly converting X-ray detector, the incident X-rays are converted into visible light in a scintillator and then in turn converted into electrical charge in photodiodes of the active matrix. This charge is stored and read out. The X-ray images may subsequently be post-processed. In order to obtain high quality X-ray images, some of the effects caused, for example, due to the specific properties of the respective X-ray detector, are to be corrected electronically, such as offset corrections, in which the dark current is corrected, and gain corrections that compensate for sensitivity variations of the X-ray detector.

After illuminating X-ray detectors with a high X-ray dose (e.g., in collimated digital subtraction angiography (DSA)), a temporary local change to the scintillator may occur at the location of the illumination. This change, known as bright-burn, results in a change to the scintillator's gain (e.g., different light yield: X-ray dose proportionality factor) and results in bright-burn artifacts in X-ray images taken thereafter (2D or reconstructed 3D X-ray images). Depending on the scintillator, the decay time of a local gain variation is between a few minutes and several days.

The prior art already contains suggested solutions for the suppression of these artifacts. For example, a so-called reset light is integrated in the X-ray detector to emit light, for example, in the blue or ultraviolet spectral region onto the scintillator/matrix before a new X-ray image is recorded. This is described, for example, in the dissertation “Physics-Based Optimization of Image Quality in 3D X-ray Flat-Panel Cone-Beam Imaging”, Rudolph Maria Snoeren, 2012, Pages 111 et seq. A further suggestion envisages calculating the gain variation from the X-ray images taken. This is known, for example, from the patent U.S. Pat. No. 7,881,555 B2.

### SUMMARY AND DETAILED DESCRIPTION

The scope of the present invention is defined solely by the appended claims and is not affected to any degree by the statements within this summary.

A simple and inexpensive possibility is provided for removing bright-burn artifacts from X-ray images. An X-ray device suitable for carrying out the method is provided.

One embodiment for processing an X-ray image of an object under examination taken with an X-ray detector with a plurality of pixel sensors to remove bright-burn artifacts includes the following acts:

provision of at least one substantially artifact-free offset image of the X-ray detector taken without the application of X-rays,

recording of at least one dark image with the X-ray detector without the application of X-rays,

correction of the at least one dark image by the at least one artifact-free offset image for determining at least one offset-corrected first correction image with afterglow artifacts,

calculation of at least one second correction image for correcting bright-burn artifacts from the first correction image with afterglow artifacts using a, for example predetermined, correlation between afterglow artifacts and bright-burn artifacts,

recording of at least one X-ray image of an object under examination with the X-ray detector, and

correction of the at least one X-ray image with the at least one second correction image to remove bright-burn artifacts.

With the method, a second correction is created in a simple way and without the use of X-rays with which bright-burn artifacts may be removed quickly and particularly effectively from X-ray images. Here, the method skillfully makes use of the correlation between afterglow-artifacts and bright-burn artifacts. Intense irradiation of the scintillator with X-rays not only causes the temporary gain variation (i.e., bright-burn) in future X-ray images, but the scintillator also temporarily produces light at the irradiation points, the so-called afterglow. Unlike bright-burn artifacts, artifacts formed from the afterglow are easy to measure by the creation of a dark image without X-rays and correction of the dark image with an offset image. As a result, with the aid of the connection between the afterglow and the bright-burn, it is then possible to calculate a second correction image for correcting bright-burn artifacts. Unlike the case with reset light irradiation of the scintillator, wherein a reset light module has to be integrated in the X-ray detector, no additional hardware is needed. With knowledge of the correlation, the method may also be performed very quickly and simply. It is advantageous to perform the creation of the second correction image and the recording of the X-ray image quickly in sequence in order to avoid a different decay stage of the artifacts on the creation of the images and hence an error in the correction.

According to one embodiment, a plurality of two-dimensional X-ray images are taken from different projection directions with respect to the object under examination and corrected by the second correction image and reconstructed to form a three-dimensional X-ray image of the object under examination.

According to a further embodiment, the at least one artifact-free offset image is taken following an operational pause of the X-ray detector of at least 12 hours, in particular at least 24 hours or at least 72 hours. Offset images are created without the application of X-rays to determine the noise of an X-ray detector. An artifact-free offset image of this kind may, for example, also be created during the operation of the X-ray detector or following a longer period of non-usage of the X-ray detector.

According to a further embodiment, the relationship between afterglow artifacts and bright-burn artifacts of the X-ray detector used is determined by a preceding method, a type of “calibration”, pixel-by-pixel (e.g., once) by measurements. The correlation may be individual for the respective X-ray detector or be dependent on the manufacture of the X-ray dector. It is possible during the preceding method, for example after planned intense irradiation of the X-ray detector, to measure afterglow and bright-burn artifacts and the decay thereof at different times following the irradiation and to calculate, determine or estimate the correlation for each pixel sensor therefrom. The functional correlation is then stored for each pixel sensor and used correspondingly for the method. Calibration of this kind may take place once or be repeated at regular intervals.

Another embodiment includes an X-ray device for carrying out the method. The X-ray device is an indirectly-converting X-ray detector with a plurality of pixel sensors, an image-processing unit for processing images taken by the X-ray detector, a calculating unit for calculating correction images, a storage unit for storing images and the correlation algorithm, and a control unit for controlling the method.

According to a further embodiment, the X-ray detector includes a scintillator and in each pixel sensor at least one photodiode. The pixel sensors include crystalline silicon as a substrate material. An X-ray detector of this kind made of crystalline silicon has the advantage that afterglow artifacts are easy to measure due to the good signal/noise ratio.

### DESCRIPTION OF THE DRAWINGS

Further advantageous embodiments are explained more detail below with reference to exemplary embodiments represented schematically in the drawings, without thereby restricting the invention to said exemplary embodiments. The drawing shows:

FIG. 1 shows a sequence of the acts of a method for processing an image to remove bright-burn artifacts according to one embodiment, and

FIG. 2 shows a view of an X-ray device of one embodiment.

### DETAILED DESCRIPTION OF THE PREFERRED EMBODIMENTS

The temporary local change of the scintillator that occurs following an illumination of X-ray detectors with a high X-ray dose may cause artifacts. Overall, the artifacts develop due to electron-hole pairs, which are generated by the X-rays and are captured in the vicinity of defects in the crystal, for example the scintillator and are released again, not immediately, but only gradually. Correspondingly, only then do the pairs generate a signal and hence impair the sensitivity of the X-ray detector. This reduces the quality of X-ray images since so-called ghost images of previous images appear on the X-ray images. During the signal processing, the artifacts have two components: an additive residual signal (afterglow) and a multiplicative gain variation (bright-burn). Both these effects are temporary and have a correlation with one another that may be measured or determined individually for each detector.

For the purposes of some embodiments, the correlation is used to remove bright-burn artifacts from X-ray images. However, the method is in particular no longer very advisable if an X-ray detector is used frequently and/or irradiation with intense doses of X-rays is applied.

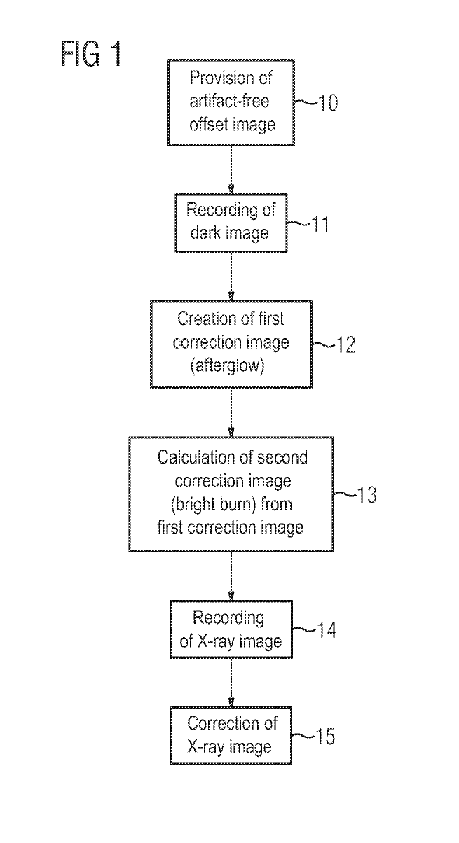

FIG. 1 shows a sequence of acts of the method. In a first act 10, a substantially artifact-free offset image of the X-ray detector taken without the application of X-rays is provided. An artifact-free offset image of this kind is preferably stored in a storage unit and retrieved from the storage unit at the start of the method. The artifact-free offset image may be taken in a calibration method or during the operation of the X-ray detector, wherein regular regeneration and storage of an offset image of this kind may be provided. The offset image may also be an offset image averaged from a plurality of offset images. To ensure that an offset image of this kind is substantially artifact-free, a rest pause before the X-ray detector recording of at least 12 hours, preferably at least 24 or 78 or 168 hours, in which no X-rays were applied to the X-ray detector, is advantageous.

In a second act 11, a dark image is taken with the X-ray detector without the application of X-rays. This dark image may also be averaged (e.g., simple averaging from a plurality of dark images). A dark image of this kind displays afterglow artifacts due to the irradiation of the scintillator, however since no X-rays are applied, the dark image does not display any bright-burn artifacts. In a third act 12, the dark image is corrected by the artifact-free offset image (e.g., by subtracting the offset image) so that a first correction image that only shows the afterglow artifact or artifacts is obtained.

In a fourth act 13, then a second correction image is created from the first correction image using the correlation, which was for example, previously determined and stored or retrieved in some other way and made available (e.g., by individual functions for each pixel sensor), between afterglow-artifacts and bright-burn artifacts in particular pixel-by-pixel, for example by calculation. The second correction image is embodied such that, when applied with a corresponding correction operation to an X-ray image (e.g., multiplicatively), the second correction image may bring about the removal of bright-burn artifacts from the X-ray image, for example by gain variation.

Following the creation of the second correction image, (in particular soon thereafter) in a fifth act 14, an X-ray image or a series of X-ray images of an object under examination are taken with the application of X-rays and, in a sixth act 15, corrected by the second correction image with a corresponding mathematical operation. In this way, it is possible to remove bright-burn artifacts from the X-ray image or images simply and effectively. The series of X-ray images may, for example, be a series of fluoroscopy images, or a plurality of projection X-ray images may be taken from different projection directions around the object under examination, corrected and then reconstructed to form a 3D volume image. The X-ray images may also be subjected to further correction operations, for example offset correction (generally before the bright-burn-correction), gain-correction (performed before or after the bright-burn-correction) and/or correction of the afterglow artifacts. It is also possible to provide further image processing acts for the X-ray images.

The relationship between the afterglow artifacts and the bright-burn artifacts of the X-ray detector used may be determined in the preceding method, a type of “calibration”, by measurements for each pixel. This may, for example, be performed once during the operation of the X-ray detector (e.g., only once). The correlation may be different for each individual X-ray detector or, depending on the manufacture, the same for a plurality of X-ray detectors. During the preceding method, it is possible, for example after targeted intense irradiation of the X-ray detector, for afterglow and bright-burn artifacts and the decay thereof to be measured at different times after the irradiation and the correlation for each pixel sensor calculated, determined or estimated therefrom. The functional correlation is then stored for each pixel sensor and used correspondingly for the method. Calibration of this kind may be performed once or repeated at regular intervals.

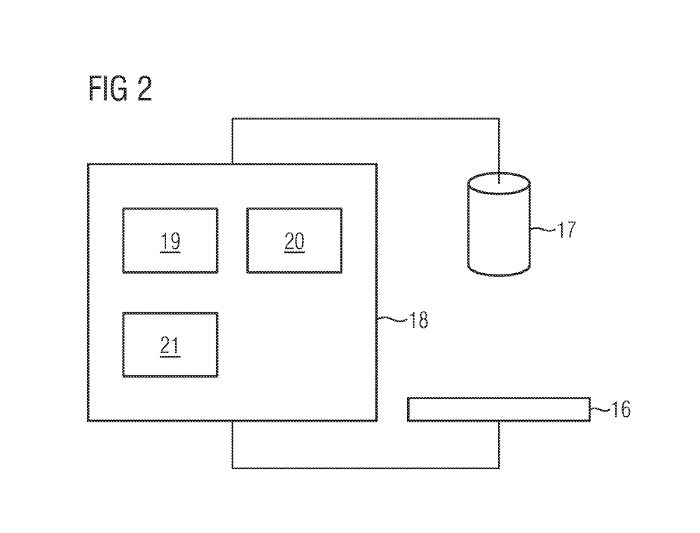

A suitable X-ray device for carrying out the method is shown, for example, in FIG. 2. The X-ray device includes an X-ray detector 16 and an X-ray source 17 for the emission of X-rays. The X-ray detector 16 and X-ray source 17 may, for example, be arranged jointly on an adjustable C-arm. The X-ray detector 16 is, for example, a digital X-ray detector with a scintillator for converting X-rays into light and with a matrix including a plurality of pixel sensors. Each pixel sensor has at least one photodiode for converting light into electrical charge. The X-ray detector may, for example, be crystalline silicon as a substrate material. An X-ray detector of this kind made of crystalline silicon has the advantage that, due to the good signal/noise ratio, afterglow artifacts are very simple to measure. However, the substrate can also be made of amorphous silicon. The X-ray device also includes a control device 18 for controlling the method. The control device 18 may be formed from a system control of the X-ray device. Also provided are an image processing unit 19, a calculating unit 20 and a storage unit 21. The image processing unit 19 is, for example, embodied to create an X-ray image from data of the X-ray detector, process the image and to make corrections to the X-ray image. The calculating unit 20 is configured to carry out calculations, and the storage unit 21 is configured to store data and X-ray images.

One embodiment may be briefly summarized as follows: for a particularly simple and effective correction of X-ray images, a method is provided for processing images of an X-ray image of an object under examination taken with an X-ray detector with a plurality of pixel sensors to remove bright-burn artifacts with the following steps: provision of at least one substantially artifact-free offset image of the X-ray detector taken without the application of X-rays, recording of at least one dark image with the X-ray detector without the application of X-rays, correction of the at least one dark image by the at least one artifact-free offset image for determining at least one offset-corrected first correction image with “afterglow artifacts”, calculation of at least one second correction image for correcting bright-burn artifacts from the first correction image with afterglow artifacts using a, for example predetermined, correlation between afterglow artifacts and bright-burn artifacts, recording of at least one X-ray image of an object under examination with the X-ray detector, and correction of the at least one X-ray image with the at least one second correction image to remove bright-burn artifacts.

Although the invention was illustrated and described in detail by the preferred exemplary embodiments, the invention is not restricted by the disclosed examples and other variations may be derived herefrom by the person skilled in the art without departing from the scope of protection of the invention.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for processing an X-ray image of an object under examination recorded with an X-ray detector with a plurality of pixel sensors to remove bright-burn artifacts, the method comprising:
providing at least one substantially artifact-free offset image of the X-ray detector taken without the application of X-rays,
recording at least one dark image with the X-ray detector without the application of X-rays,
correcting of the at least one dark image by the at least one artifact-free offset image for determining at least one offset-corrected first correction image with afterglow artifacts,
calculating at least one second correction image for correcting bright-burn artifacts from the first correction image with afterglow artifacts using a correlation between the afterglow artifacts and the bright-burn artifacts,
recording of at least one X-ray image of an object under examination with the X-ray detector, and
correcting the at least one X-ray image with the at least one second correction image to remove bright-burn artifacts.

2. The method as claimed in claim 1, further comprising:
taking a plurality of two-dimensional X-ray images from different projection directions with respect to the object under examination,
correcting the two-dimensional X-ray images with the second correction image, and
reconstructing the corrected two-dimensional X-ray images to form a three-dimensional X-ray image.

3. The method as claimed in claim 1, wherein the at least one artifact-free offset image is taken following an operational pause of the X-ray detector of at least 12 hours.

4. The method as claimed in claim 3 wherein the pause is at least 24 hours.

5. The method as claimed in claim 3 wherein the pause is at least 72 hours.

6. The method as claimed in claim 1, wherein the relationship between the afterglow artifacts and the bright-burn artifacts of the X-ray detector used is determined pixel-by-pixel by measurements.

7. The method as claimed in claim 6 wherein the relationship is determined only once.

8. The method as claimed in claim 2, wherein the at least one artifact-free offset image is taken following an operational pause of the X-ray detector of at least 12 hours.

9. The method as claimed in claim 2, wherein the relationship between the afterglow artifacts and the bright-burn artifacts of the X-ray detector used is determined pixel-by-pixel by measurements.

10. The method as claimed in claim 3, wherein the relationship between the afterglow artifacts and the bright-burn artifacts of the X-ray detector used is determined pixel-by-pixel by measurements.

11. The method as claimed in claim 1 wherein calculating uses the correlation as a predetermined correlation.

12. An X-ray device comprising:
an indirectly-converting X-ray detector with a plurality of pixel sensors, the detector configured to take at least one substantially artifact-free offset image without the application of X-rays and at least one dark image without the application of X-rays,
an image-processing unit configured to process images taken by the X-ray detector,
a calculation unit configured to calculate correction images, the at least one dark image corrected by the at least one artifact-free offset image as at least one offset-corrected first correction image with afterglow artifacts, at least one second correction image for correcting bright-burn artifacts from the first correction image with afterglow artifacts using a correlation between the afterglow artifacts and the bright-burn artifacts,
a storage unit configured to store at least one X-ray image of an object under examination with the X-ray detector, and
a control unit configured to correct the at least one X-ray image with the at least one second correction image to remove bright-burn artifacts.

13. The X-ray device as claimed in claim 12, wherein the X-ray detector comprises a scintillator and at least one photodiode in each pixel sensor, and wherein the pixel sensors comprise crystalline silicon as a substrate material.
